(19) United States
(12) Patent Application Publication
(10) Pub. No.: US 2004/0224343 A1
(43) Pub. Date: Nov. 11, 2004

(54) Methods and products to enhance interactions among molecules

(75) Inventors: Xiaoliang Han, San Francisco, CA (US); Yibing Yang, Fremont, CA (US)

Correspondence Address: C. P. Chang, c/o Pacific Law Group LLP, Suite 525, 224 Airport Parkway, San Jose, CA, 95110, US

(73) Assignee: BioChain Inc., Hayward, CA (US)

(21) Appl. No.: 10/839,843

(22) Filed: May 5, 2004

Related U.S. Application Data
(60) Provisional application No. 60/468,489, filed on May 6, 2003.

(51) Int. Cl. C12Q 1/68; G01N 33/53; C12P 21/06

(52) U.S. Cl. 435/6; 435/7.1

(57) ABSTRACT

Treatments of molecules with organic chemicals or bipolar reagents are used to enhance the interactions among or between molecules such as binding between proteins and antibodies or between nuclear acids. Such organic chemicals or bipolar reagents include alcohols, detergent, ester and ether. Treatments of molecules with the organic chemicals or bipolar reagents can take place before or during interactions among or between molecules. Enhancing intensities are about 2 to 100 times depending on molecules, organic chemicals or bipolar reagents and timing to treat molecules.

### REFERENCE TO RELATED APPLICATION

This application claims the benefit of a provisional application filed May 6, 2003, Ser. No, 60/468,489 by the same inventors.

### FIELD OF THE INVENTION

This invention relates to a method and products for enhancing the interactions of molecules. In particular, this invention relates to a method and products for enhancing the abilities of molecules such as proteins, antibodies and nuclear acids to interact between and among each other in an optimized reaction environment applicable to biomedical research and diagnosis of diseases.

### BACKGROUND OF THE INVENTION

Abilities of the interactions among molecules, such as molecules binding to each other are very important features of molecules, such as binding between proteins and antibodies or between nuclear acids. Enhancing abilities of the interactions among molecules will have great effects on application of binding between molecules in biomedical research and diagnosis of diseases. Many factors determine the interactions among molecules, such as the structures of molecules themselves and different conditions for interactions among molecules. These factors can be modified into optimized conditions for interactions among molecules. Ionic strength and pH have been known as key factors for interactions among molecules. This invention has discovered that organ chemicals can enhance the interactions among molecules under optimized conditions in ionic strength and pH.

### SUMMARY OF THE INVENTION

One aspect of the invention is to provide a method for enhancing interactions among or between molecules in a reaction environment that comprises the steps of: providing the molecules with organic chemicals or bipolar reagents in the reaction environment; and treating the molecules with the organic chemicals or bipolar reagents for a period of time sufficient to induce or enhance interactions among said molecules. The molecules according to the invention comprise a series of cellular molecules capable of interacting or binding to each other, such as proteins and antibodies, or nuclear acid and nuclear acid.

One more aspect of the invention is to provide for a variety of organic chemicals or bipolar reagents which are chemicals containing at least carbon and hydrogen and showing function of enhancing interactions among or between molecules, such as alcohols, detergents, esters, and ethers. The treatments of the molecules by organic chemicals or bipolar reagents can take place before the interactions among or between the molecules, or during interactions among or between molecules. The method of treatment further comprises the step of treating the molecules to be interacted with other counterpart molecules. Furthermore, the molecules can be in a denatured or in a native status or be immobilized on supporting materials such as in immunoprecipitation, protein crystallization and flow cytometry or applicable to electrical transfer, capillary transfer, direct spotting, or fixation. In practice, the supporting materials can be made into different shapes, formats or status, such as membrane, multiple well plates, sheet, fiber, column, beads, and glass plate and from organic or non-organic chemicals, such as nylon, nitrocellulose, polyvinylidene difluoride (PVDF), polyvinyl chloride (PVC), plastic, silicon, and natural biological materials. In addition, the step of treating the molecules by organic chemicals or bipolar reagents includes treatment of the supporting materials to enhance interactions among or between molecules.

According to the invention, the alcohols showing function of enhancing interactions among or between molecules are methanol, ethanol, 2-propanol, 1-butanol, IsoAmyl alchohol, and 1-octanol. The detergent showing function of enhancing interactions among or between molecules at least is Tween 20. The ester showing function of enhancing interactions among or between molecules at least is Tween 20 and the ether at least is Dimethylsulfoxide (DMSO).

According to the invention, the step of treating the molecules with organic chemicals or bipolar reagents before or during the interactions among or between molecules includes immersing the molecules into organic chemicals or bipolar reagents, or into a media containing organic chemicals or bipolar reagents for 1 second to 10 minutes or for an appropriate time. The organic chemicals or bipolar reagents used in treatments can be a single organic chemical or a bipolar reagent, or can be mixture of organic chemicals or bipolar reagents. The organic chemicals or bipolar reagents can be 100% pure, or can be a mixture in any percentage with other chemicals or water. After the treatment, the molecules can interact with other counterpart molecules such as antibodies and nuclear acids. The antibodies can be monoclonal or polyclonal antibodies and derived at least from the hosts of mouse or rabbit. The immunogens or antigens used to raise the antibodies can be at least peptides or proteins or from nuclear acids-such as DNA or RNA and the antibodies can bind preferably to either native antigens or denatured antigens.

According to the method of the invention, the step of enhancing interactions among or between molecules with treatments of organic chemicals or bipolar reagents includes reconfiguring structures of epitopes on molecules, unmasking noncovalent bonds between molecules, or restoring charges, hydrophilic or hydrophobic status of molecules. The organic chemicals or bipolar reagents can be 100% pure, or can be mixture in any percentage with other chemicals or water.

One additional aspect of the invention is to provide for a product for enhancing interactions among or between molecules in a reaction environment that comprises a) suitable organic chemicals or bipolar reagents prepared in the reaction environment; and b) molecules of interest added to said organic chemicals or bipolar reagents for treatment for a period of time sufficient to induce or enhance interactions among said molecules of interest.

### BRIEF DESCRIPTION OF DRAWINGS AND FIGURES

FIG. 1 Concentration Titration and Comparison of Western on PVDF Membranes Treated with Different Alcohols

Human normal placenta total proteins were loaded on SDS-PAGE with one wide well and transferred on to PVDF membrane. PVDF membrane was cut into stripes and each strip contains about 2 ug of protein. Each stripe was soaked in different concentration of alcohol solution before the blocking step. Results of Western blot were included in Panel A. B, and C. A mouse anti-human GAPDH antibody was used as the primary antibody. A single band around the 37.4 kDa size marker, which represent the human GAPDH protein, was observed on each stripe. The strength of signal was alcohol type and concentration dependent. Generally a 1 to 9-fold increase of signal could be obtained by treatment with each alcohol under its optimal concentration. The concentrations of alcohols, under which shown enhancing effect, range from 1% to 100%.

FIG. 2 Pre-treatment and in buffer System Treatment of Western blot on PVDF Membranes with Alcohols or Ether

Human normal placenta total proteins were loaded on SDS-PAGE with one wide well and transferred on to PVDF membrane. PVDF membrane was cut into stripes and each strip contains about 20 ug of protein. Each stripe was soaked in different concentration of organic chemicals before or during first antibodies binding as pre-treatment or in buffer System treatment. Pretreatment with alcohols can enhance interactions between molecules. 1-octanol and isoAmyl alcohol enhance more than other alcohols. 1-octanol can enhance interactions between molecules about 100 times than control. Both antibodies anti GAPDH and Actin have similar result. Pretreatment with ether such as DMSO can enhance interactions between molecules also. Treatment in buffer System by alcohols or ether can enhance interactions between molecules with less capability than pretreatment.

FIG. 3 Comparison of Western Blotting Signals in Different Detergent Buffer Systems

Human normal placenta total proteins were loaded-on SDS-PAGE with one wide well and transferred on to PVDF membrane. PVDF membrane was cut into stripes and each strip contains about 2 μg of protein. PVDF stripes were tested for western blot in five different detergent buffer systems and an aromatic buffer system. Different concentration points were chosen for each system. The signals increased 0.2 to 19 fold in all detergent buffer systems except the SDS buffer system, comparing to buffer system without any detergent. Tween-20 is a far more potent enhancer than other detergents. Briji-35 is the second potent enhancer. NP-40 and Triton-100, which have similar molecular structure, have no significant influence on the signal.

FIG. 4 Effect of Milk, Pre-treatment, Buffer system, and Tween-20 Concentration on Western Signal Noise Ratio

Human normal placenta total proteins were loaded on SDS-PAGE with one wide well and transferred on to PVDF membrane. PVDF membrane was cut into stripes and each strip contains about 2 μg of protein. PVDF stripes were tested for western blot by alcohol pre-treatment or buffer system using a chicken anti-human GAPDH antibody 1:2000 (Panel A) and rabbit anti-human EGFR antibody 1:200 as primary antibodies (Panel B). In Panel A, the nonfat milk used was from Safeway. The chicken poly-clonal antibody has non-specific bindings and very high background. There wasn't much difference between un-blocked (the left five lanes) and blocked blots (the middle five lanes). The right five lanes, was blot which had been blocked and included milk in primary and secondary antibodies solutions, however, had the highest background. Pre-treatment and higher concentration of Tween-20 in buffer dramatically decreased background, and increased signal noise ratio (lanes 5, 10 and 15). Lane 5 had the lowest background, suggested under certain circumstances, pre-treatment can substitute blocking step. In panel B, the anti-EGFR antibody was not very sensitive, so excess amount of A431 lysates was loaded in the gel. A portion of EGFR protein was degraded. The nonfat milk used was from Bio-Rad; the blot with blocking and milk in antibody solutions had the lowest background. The result made more sense compared to Panel A, this told us that the kind of milk used matters for the blot very much. Pre-treatment also decreased background in the right 10 lanes.

FIG. 5 Effect of Tween-20 and Pre-treatment by Different Alcohol on Western Signal

Human normal placenta total proteins were loaded on SDS-PAGE with one wide well and transferred on to PVDF membrane. PVDF membrane was cut into stripes and each strip contains about 2 μg of protein. Mouse Anti-GAPDH 1:2000 as primary antibody was in 1×TTBS or 1×TBS. The secondary antibody was in 1×TTBS (0.1% TW) buffer. Different stripes were pre-treated by different alcohols. All of the tested pre-treatments enhanced Western signal, to different extent. Pre-treatments with 20% iso-amyl alcohol and 70% isopropanol had the biggest enhancement, indicating alcohols containing iso-carbon-chain are favored enhancers. Without Tween-20 in the buffer (lanes 1 and 10), the background increased dramatically.

FIG. 6 Effect of Tween-20 concentration in primary antibody solution on Western signal

Human normal placenta total proteins were loaded on SDS-PAGE with one wide well and transferred on to PVDF membrane. PVDF membrane was cut into stripes and each strip contains about 2 μg of protein. Mouse Anti-GAPDH 1:2000 as primary antibody was in buffers containing different concentrations of Tween-20. Secondary antibody was in 1×TTBS (0.1% TW) buffer. The optimal Tween-20 concentration in the primary antibody solution was between 0.5% and 1.5%. Pre-treatment did enhance the Western signal (lanes 12 and 13).

FIG. 7 Effect of Tween-20 concentration in secondary antibody solution on Western signal Human normal placenta total proteins were loaded on SDS-PAGE with one wide well and transferred on to PVDF membrane. PVDF membrane was cut into stripes and each strip contains about 2 μg of protein. Mouse Anti-GAPDH 1:2000 as primary antibody was in 1×TTBS (0.1% TW) buffer. The Secondary antibody was in buffers containing different concentrations of Tween-20. The optimal Tween-20 concentration in secondary antibody buffer was around 1%. Pre-treatment did enhance the signal (lanes 8, 9).

FIG. 8 Comparison of Western on PVDF with Alcohol or DiMethylSulfOxide (DMSO) Pre-treatment or in Alcohol or DMSO Buffer System

Human normal placenta total proteins were loaded on SDS-PAGE with one wide well and transferred on to PVDF membrane. PVDF membrane was cut into stripes and each strip contains about 2 μg of protein. Stripes of PVDF membrane were tested for western blot by alcohol or DMSO pre-treatment, or in alcohol or DMSO buffer system. The Tween-20 concentration was 0.05%. The signal increased 1 to 2 fold by DMSO pre-treatment, and 2 to 7 fold in DMSO buffer system.

FIG. 9 Comparison of Western on Nitrocellulose or Hybond N+ Membrane Pre-treated with Alcohols or with Alcohols in Buffer System

Human muscle total proteins were loaded on SDS-PAGE with one wide well and transferred on to Nitrocellulose membrane and Hybond N+ membrane. Nitrocellulose membrane and Hybond N+ membrane was cut into stripes and each strip contains about 2 μg of protein. Stripes from Nitrocellulose membrane, and Hybond N+ membrane were tested in pre-treatment with two different types of alcohol, or in buffer systems containing these two types of alcohol. A 10 to 30 fold sharp increase of signal was observed for Hybond N+ membrane pre-treated with alcohols.

FIG. 10 Comparison of Western blots on PVDF or Nitrocellulose Membrane in the presence of different Tween-20 concentrations with Alcohol Pre-treatment or Buffer System

Human normal placenta total proteins were loaded on SDS-PAGE with one wide well and transferred on to PVDF (Panel A) or Nitrocellulose (Panel B) membrane. PVDF (Panel A) or Nitrocellulose (Panel B) membrane was cut into stripes and each strip contains about 2 μg of protein. The stripes were incubated with Mouse anti-GAPDH antibody. Pre-treatment enhanced signal significantly in both cases (19 folds on PVDF, and 9 folds on NC), whereas buffer system only worked on PVDF membrane.

FIG. 11 Enhancement of Western Blot Detected with A Mouse Anti-human Actin Antibody

Human normal placenta total proteins were loaded on SDS-PAGE with one wide well and transferred on to PVDF membrane. PVDF membrane was cut into stripes and each strip contains about 5 μg of protein. PVDF stripes were tested for western blot by alcohol pre-treatment or buffer system using a mouse anti-human actin monoclonal antibody. The signal was increased 0.2 to 4 fold under two different circumstances, indicating the enhancing effect on Western blot was not limited to Anti-GAPDH antibody only. Interestingly, blot (lanes 4-6) blocked with non-fat milk (from Safeway) had stronger signal comparing to the blot without any blocking (lanes 1-3).

FIG. 12 Enhancement of Western Signals Against Three Different Antigens on the Same PVDFMembrane Pre-treatment with 20% Iso Amyl-alcohol (ISA)

Four pieces of similar amount of rat liver tissue were used for compartment protein extraction. The C buffer and TM buffer were made with traditional formula. The N buffer used contains 2%, 5%, 10% and 25% glycerol, respectively. M2, M5, M10 and M25 stand for the membrane fractions from each extraction. A 80-fold increase in western signal blotted against GAPDH, a 8-fold increase in blot against Beta-actin, and a 23-fold enhancement in blot against EGFR were observed upon pre-treatment with 20% ISA.

FIG. 13 Comparison of Protein Dot Blots on Different Membranes in the Presence or Absence of Alcohol in Buffer System

Indicated concentrations of human normal muscle total proteins were coated on PVDF, or Nitrocellulose, or Hybond N+ membranes using an array pin, and blotted with mouse anti-GAPDH antibody. For samples on PVDF membrane, 5% of Iso-amyl Alcohol (ISA) was included to assist adhesion. Two pieces of each type of membrane, which were coated with the same amount of serial diluted proteins, were used for immunoassays. One was in normal buffer system, and the other in buffer containing 0.05% ISA. The signal increased in buffer system by 7 to 14 fold on PVDF membrane, 1 to 4 fold on Nitrocellulose membrane, and 3 fold on Hybond N+ membrane.

FIG. 14 Comparison of Protein Dot Blots on Nitrocellulose, Hybond N+, and Hybond Neutral Membranes with or without Pre-treatment

Indicated concentrations of human normal muscle total proteins were coated on Nitrocellulose, Hybond N+ and Hybond Neutral membranes using an array pin, and blotted with mouse anti-GAPDH antibody. Pre-treatment enhanced protein dot blot signals on all three membranes. A 4 fold-increase and a 1 fold-increase were observed on NC and Hybond Neutral membrane, respectively.

FIG. 15 Enhancement of Human Normal Tissue Protein Arrays on Nitrocellulose and Hybond Neutral Membranes by Pre-treatment

Thirty two-tissue duplicated-spots protein arrays on Hybond Neutral and NC membranes were used to test pre-treatment enhancement. Signals on both membranes had significant enhancement after pre-treatment.

FIG. 16 Comparison of Dot Blots of Proteins Prepared by TM Buffer and XO Buffer

Total proteins were extracted from human normal tissues using lysis buffer containing 0.5% NP-40 (TM) or 0.5% SDS (XO). A panel of proteins in different lysis buffers was coated on four Nitrocellulose (NC) and four Hybond Neutral (HN) membranes, respectively. Two pieces of each membrane were pre-treated with 20% iso-amyl alcohol (ISA), and the other two pieces were untreated. One pair of treated, untreated NC and HN membranes were blotted with mouse anti-actin antibody, another pair of each membrane were blotted with mouse anti-GAPDH antibody. Signals of Proteins in TM buffer blotted with GAPDH antibody were enhanced many folds with 20% ISA pre-treatment, on either NC or HN membrane. Whereas the signals of Proteins in TM buffer blotted with Actin antibody were enhanced with 20% ISA pre-treatment, only on HN membrane, not on NC membrane.

FIG. 17 Effects of Buffer Systems on RNA Dot Blots

Indicated concentrations of mouse muscle mRNA were coated on four pieces of Hybond H+ membrane, and hybridized with C19 beta-actin probe. Membranes in Panel A, B, C, and D were hybridized in normal hybridization buffer, hybridization buffer containing 0.05% Iso-amyl Alcohol, hybridization buffer containing 2% DMSO, hybridization buffer containing 2% methanol, respectively. The signal increased 1 to 2 fold in both the alcohol and ether buffer systems comparing to in normal buffer.

FIG. 18 RNA Dot Blots on Hybond N+ and Hybond Neutral Membranes with or without 20% ISA Pre-treatment

Indicated concentrations of mouse muscle mRNA were coated on two pieces of Hybond H+ and two pieces of Hybond Neutral membranes, and hybridized with C19 beta-actin probe. One piece of each type of membrane was untreated, the other piece was pre-treated with 20% iso-amyl Alcohol. On Hybond N+ membrane, a two-fold increase in signal was reached.

FIG. 19 Enhancement in Northern blot Analysis and ELISA by pretreatment

Hybridization of Northern blot analysis of human normal heart tissue is performed with actin probe as shown in Panel A. Lane 1 is the result without pre-treatment and lane 2 is the result with pre-treatment by 20% IsoAmyl alcohol. Pre-treatment increases signal intensity about 3 folds. Panel B shows enhancement in ELISA by pretreatment with isopropanol. Lane 1, 2 and 3 are GAPDH protein coated in multiple wells at concentration 20, 2 and 0.2 ng/ul respectively. Row F is Non-treatment control and row G and H are pretreated with isopropanol.

FIG. 20 Enhancement in Immunohitochemistry Analysis of Tissue section on Glass Slide by pretreatment

Immunohitochemistry Analysis of breast cancer tissue sections is performed with P53 antibody. Panel A is the result without pre-treatment and Panel B is the result with pre-treatment by 20% IsoAmyl alcohol. Pre-treatment increases signal intensity as arrow pointed.

### DETAILED DESCRIPTION OF THE INVENTION

This invention discovers a novel method to increase or enhance abilities of interactions between molecules, especially biomolecules, such as proteins or nuclear acid, by optimizing environment condition for interactions. Interactions between molecules are determined by the intrinsic bonds (the properties of components on molecules, distance between components, or conformational structures of epitopes), valences between molecules, three-dimensional structure of multiple binding sites on molecules to form multimeric complexes. The properties of components on molecules, potential valences between molecules and multiple binding sites on molecules are given by nature in the molecules, but optimization in environment condition for interactions such as hydrophilic or hydrophobic conditions by organic chemicals or bipolar reagents may optimize distance between components on molecules, conformational structures of epitopes and three-dimensional structure of multiple binding sites on molecules, therefore increase capabilities of interactions between molecules.

Interactions between molecules are the most applicable principles in life science discovery, diagnosis and therapies of disorder in living beings, including human, animal and plants. The most applied molecules in life science are proteins, amino acid, antibodies, nuclear acid, lipid, carbohydrates, and synthetic organic chemicals. There are many different nomenclatures to describe the interactions between molecules. Interaction between antigen and antibodies is usually called as antibodies binding to antigen that can be any one or complexes of above molecules. Term of protein and protein interaction is adopted in cellular signal transductions. Transcription factors are called as DNA binding proteins that interact with DNA. Interactions between nuclear acids including DNA and RNA are described as hybridization. Antibodies interacted with proteins on tissue sections are called as immunol staining or immunohistochemistry. Interaction is a broad definition of interacting activity between molecules, while binding is used frequently in interacting activity between antibody and antigen.

There are many forces to facilitate interactions between molecules. The major forces are multiple, noncovalent bonds, including hydrogen bonds, van der Waals force, coulombic interaction and hydrophobic bonds. In antibody and antigen interaction, these bonds formed between side chains or polypeptide backbones. The bonds usually formed between the components of molecules. A component is a subunit of molecules, such as a methyl group on an amino acid. Properties of components on molecules and distance between components determine what or whether the bonds are formed. Conformational structures of molecules determine where and how the bonds were formed and tightness of these bonds. Environments such as pH, ionic strength, hydrophilic or hydrophobic conditions and other unmentioned variances will change the properties of components on molecules, distance between components, or conformational structures of molecules, thus affect the formation of bonds between molecules and interactions between molecules.

Interactions or bindings between molecules are mediated through epitopes. Epitopes forms the unique site for interaction between molecules, such as binding between antigen and antibody. As binding sites, the epitopes can be amino acid, carbohydrates, lipid, nuclear acid or synthetic organic chemicals. The binding sites can be a motif with charged or hydrophobic molecules. The motif consists of a single or multiple components, such as a single or a group of amino acid. The geometric arrangement of interaction components or three-dimensional structure of epitopes determines uniqueness of the motif on an epitopes. As most of epitopes forms very unique binding site, the interactions between molecules are specific. The degree of specificity of binding between molecules is one parameter to evaluate quality of binding between molecules. The more specific the binding is between molecules, the more valuable in application.

Other important parameter to measure the interaction-or binding between molecules is affinity. Affinity indicates the binding strength between molecules when they start to bind to each other. This parameter is also known as on-binding force. Many factors determine the affinity strength, and among which the major factors are intrinsic properties of bonds, valencies between molecules, or multiple binding sites between molecules. Some bonds are stronger than other bonds under a preferred condition depending on properties of components on molecules, distance between components, or conformational structures of molecules as mentioned above; some molecules have more valences when intrinsic properties of bonds are the same, such as IgG is bivalent while IgM has 10 valences to bind to antigen; some molecules have multiple binding sites to form multimeric complexes to increase affinity strength.

Avidity is the parameter to measure the stability of complex after binding between molecules. This parameter is also known as off-binding force. It is the most important property about the interactions between molecules. It is generally agreed that high avidity is highly desirable in many research, diagnostic or therapeutic applications. The strength of avidity is determined by many factors also, but among which the major factors are the factors affecting specificity and affinity of interactions between molecules. Usually under a not optimized condition, the intrinsic bonds (the properties of components on molecules, distance between components, or conformational structures of epitopes), valences between molecules, three-dimensional structure of multiple binding sites on molecules to form multimeric complexes are inhibited, crippled or may not in their best conditions for interactions between molecules. By optimizing conditions, the inhibited intrinsic bonds and valences between molecules can be unmasked, crippled binding sites can be fixed or new binding sites can be created, which will increase or enhance the avidity strength of binding between molecules.

The common applications of interaction between molecules in life sciences discovery are Western blotting, Immunohistochemistry (IHC), Enzyme-Linked Immunosorbent Assay (ELISA), Northern blotting, Southern blotting, Microarray and etc. Western blotting, Immunohistochemistry (IHC) and Enzyme-Linked Immunosorbent Assay (ELISA) utilize the principle of specific binding (interaction) between antigens and antibodies. Northern blotting and Southern blotting depends on hybridization (interaction) of nuclear acid to nuclear acid. Microarray can be either between antigens (proteins) and antibodies, or between nuclear acids. To discover the method of enhancing or increasing binding (interaction) of molecules, representative methods such as Western blotting, microarray and Immunohistochemistry (IHC) are selected to perform this invention.

In life sciences discovery most molecules such as proteins and nuclear acids are immobilized onto supporting material made into different shapes, formats or status, such as membrane, multiple well plates, sheet, fiber, column, beads, and glass plate. The membrane itself can be made from nylon, nitrocellulose (NC), polyvinylidene difluoride (PVDF) or other materials. Multiple well plates are usually made from polyvinyl chloride (PVC) in application of ELISA, but nitrocellulose coated multiple well plate give better results. The major component in glass slide is silicon dioxide (SiO2). Natural biological materials can serve as supporting materials too, such that, for example, connective tissues serving as supporting materials for proteins in tissue sections in which glass slide may also play the supporting role. In this invention, membranes made from different materials, Multiple well plates, glass slide and tissue section are tested to prove the application of this invention on a broad range of materials.

Depending on the treatment and reaction conditions, the molecules may or may not be immobilized on to supporting materials. This invention tests some selective methods to immobilize molecules on to supporting materials, such as electrical transfer, capillary transfer, direct spotting and fixation by fixative. Proteins are electrically transferred onto supporting materials to make Western blot in present of buffer conducting electricity. Nuclear acids are transferred by capillary action onto supporting materials to make Northern blot. Proteins and nuclear acids can be directly spotted onto supporting materials. Proteins and nuclear acids can be fixed into tissues by fixative, such as formaldehyde, and tissues or tissue matrixes serve as supporting materials. When tissue sections were cut from fixed tissues or tissue matrixes, and put onto glass slide, both tissue matrixes and glass slides can serve as supporting depending on whether the proteins or nuclear acids are attached to surface of glass or cross linked to tissue matrixes, such as connective tissues, cytoskeleton structures, cellular membrane complexes or cell matrixes by fixatives. Molecules can be immobilized onto supporting materials by nature, such as proteins or nuclear acids in living cell are relatively “immobilized” by nature. In the alternative, some molecules may not need to be immobilized on to supporting materials to interact with counterpart molecules, such as immunoprecipitation proteins by antibodies, protein crystallization or flow cytometre analysis.

The example counterpart molecules used in this invention for interaction between molecules are antibodies or nuclear acid, which are the probes frequently used in life science discovery and diagnosis of disorders. Antibodies anti different antigens, such as antibodies anti cellular protein (GAPDH) from Chemicon, anti structure protein (Actin) from Sigma and anti membrane protein (EGFR) from Santa Crus Biotechnology are tested as first antibodies. These antibodies are derived from different hosts. Antibodies anti GAPDH and Actin are from mouse and antibodies anti EGFR is from rabbit. Antibodies anti GAPDH and Actin are monoclonoal antibodies and antibodies anti EGFR is polyclonal antibody. Antigens or immunogens used to raise antibody are different. GAPDH is from rabbit muscle; Actin is N-terminal peptide and EGFR is C-terminal peptide. These antibodies may have preference to binding on native proteins or denatured proteins. Secondary antibodies used in this invention are conjugated with horseradish peroxidase (HRP) or alkaline phosphatase (AP). Probe used in RNA analysis is made from human actin cDNA products amplified and labeled by polymerase chain reaction. This probe is a housekeeping gene labeled by a very common method.

Conventional methods to optimize conditions for interactions between molecules are through adjusting pH and Ionic strength in reaction environment, such as in buffer or solution. pH and Ionic strength can maintain or change charges of molecules, either positive or negative charge of components on molecules. No prior art and effort has been put on adjusting or maintaining hydrophilic or hydrophobic status of components on molecules to optimize interactions between molecules. This invention has chosen the approach to adjust or maintain hydrophilic or hydrophobic status of components on molecules to optimize interactions between molecules.

Reagents used in this invention cover a broad range of organic chemicals, including alcohols, detergents, ester, ether, hydrocarbons, proteins, organic salt, and etc. Representative alcohols tested in this invention include methanol, ethanol, 2-propanol, 1-butanol, IsoAmyl alcohol, 1-octanol, Brij 35 and Triton X-100; Representative detergents include Brij 35, NP-40, SDS, Triton X-100, and Tween 20. Representative ester is Tween 20. Representative ether is dimethylsulfoxide (DMSO). Representative hydrocarbon is xylenes. Representative proteins are bovine serum albumin and non-fat dry milk. Representative organic salt is SDS. Some chemicals have more than one property, such that Triton X-100 is detergents but also is alcohol. Detergents are more diversified and can be alcohol (Brij 35), ester (Tween 20) or organic salt (SDS). Bipolar organic reagents are preferred in the tests of this invention because bipolar organic reagents can be compatible with hydrophilic or hydrophobic components on molecules.

The molecules are treated with the organic chemicals before or during interaction (binding) with other molecules. Treatment of molecules before interaction (binding) with other molecules is called “pre-treatment of molecules” as applicable to this invention. Treatment of molecules during interaction (binding) with other molecules is called “buffer system treatment of molecules” as applicable in this invention. Pre-treatment of molecules takes 1 second to 20 minutes by applying organic chemicals onto molecules. Buffer system treatment of molecules takes the same period of time as binding of molecules by submersing molecules into buffer system containing organic chemicals in appropriate concentrations. The organic chemicals are applied either as single reagent in different concentrations or as mixture of a few reagents in an appropriate ratio. The molecules can be treated by only one organic chemical in one test or can be treated by combination of more than one organic chemical on different steps sequentially in one test.

The molecules can interact in different phases, such as liquid phase interactions or solid phase interactions. The examples of molecules interacted in liquid phase are immunoprecipitation, proteins crystallization or flow cytometre analysis. The examples of molecules interacted in solid phase are immunoassay on Western blot or protein dot blot, hybridization on Northern blot or nuclear acid dot blot, immunohistochemistry or in situ hybridization on tissue sections. This invention selects the molecules interacted in solid phase as test modes, such as immunoassay on Western blot or protein dot blot, hybridization on Northern blot or nuclear acid dot blot, immunohistochemistry on tissue sections.

Immunoassays on Western blot or protein dot blot are performed to test interaction between proteins and antibodies according to published, conventional and well acceptable method. Briefly, proteins are separated on 4-20% SDS-Polyacrylamide Gel Electrophoresis (PAGE). Proteins inside gel are immobilized on to different membranes by electric transfer or direct spotting. Pretreatment with organic chemicals for 1 second to 10 minutes is optional at this step. The membranes are incubated with 5% fat free dry milk in Tris Buffered Saline containing 0.1% Tween 20 (TTBS) to block nonspecific binding. First antibodies, such as those for GAPDH, Actin or EGFR proteins are interacted with proteins on the membrane in TTBS or TBS (Tris Buffered Saline) buffer for one hour to three days. During interactions between first antibodies and proteins, organic chemicals in an appropriate concentration are present if the buffer system treatment is applied for the same period as interactions between the first antibodies and proteins. After wash off first antibodies (and organic chemicals if applied) with TTBS, secondary antibodies conjugated with HRP or AP are incubated with membranes in TTBS or TBS for 30 to 60 minutes. After wash off secondary antibodies, membranes are incubated with substrates for HRP or AP, and then exposed to films to get black signal on films. The more interactions between antibodies and proteins on membranes, the stronger or blacker the signals are.

Hybridization on Northern blot or nuclear acids dot blot are performed to test interactions between nuclear acids according to published, conventional and well acceptable method. Briefly, nuclear acids are separated on 1% denaturing agarose gel electrophoresis (PAGE). Nuclear acids inside gel are immobilized on to different membranes by capillary transfer or direct spotting. Pretreatment with organic chemicals for 1 second to 10 minutes is optional at this step. The membranes are incubated with 7% SDS in Phosphate Buffered Saline (PBS) with 5% Bovine Serum Albumin (BSA) for 2 hour to block non-specific binding. GAPDH or Actin cDNA probes labeled with fluorescein are interacted or hybridized with nuclear acids on the membrane in 7% SDS in Phosphate Buffered Saline (PBS) with 5% Bovine Serum Albumin (BSA) for overnight, during which organic chemicals in an appropriate concentration are present if the buffer system treatment is applied for the same period as interactions between probes and nuclear acids on the membrane. After wash off first probes (and organic chemicals if applied) with buffer, anti-fluorescein antibodies conjugated with AP are incubated with membranes in TTBS or TBS for 30 to 60 minutes. After wash off anti-fluorescein antibodies, membranes are incubated with substrates for AP, and then exposed to films to get black signal on films. The more interactions between nuclear acids probe and nuclear acids on membranes, the stronger or blacker signals are.

Immunohistochemistriy of tissue section on glass slide are performed to test interaction between proteins in tissues and antibodies according to published, conventional and well acceptable method. Briefly, tissues are fixed in 4% of formaldehyde to cross link proteins or RNA to tissue matrixes for four hour to overnight. Fixed tissues are dehydrated and embedded with paraffin into paraffin embedded tissue block. Tissue sections in 5 to 10 micro millimeter were cut from tissue block and put on glass slides. Tissue sections on glass slides are de-paraffined or de-waxed in Xylenes and re-hydrated in ethanol with a serial concentration of 100%, 95% and 75%. Pretreatment of tissue sections with glass slide by organic chemicals for 1 second to 10 minutes is optional at this step. The tissue sections on glass slides are incubated in PBS buffer containing with 3% Fetal Bovine Serum (FBS) to block nonspecific binding. First antibodies, such as for p53 proteins are interacted with proteins on the tissue section in PBS buffer for one hour to overnight. After wash off first antibodies, secondary antibodies conjugated with HRP are incubated tissue sections PBS buffer for 30 to 60 minutes. After wash off secondary antibodies, tissue sections are incubated with substrates for HRP and brown color will develop in the location or target proteins. The more interactions between antibodies and proteins on tissue section, the stronger or browner signals are.

A broad selection of organic chemicals has been tested in this invention including alcohols, detergents, esters, ethers, hydrocarbons, organic salt, proteins, and etc. Alcohol examples are methanol, ethanol, 2-propanol, 1-butanol, IsoAmyl alcohol, 1-octanol, Brij 35 and Triton X-100; Representative detergents include Brij 35, NP-40, SDS, Triton X-100, and Tween 20. Ester example is Tween 20. Ether example is dimethylsulfoxide (DMSO). Hydrocarbon example is xylenes. Proteins examples are bovine serum albumin and non-fat dry milk. Organic salt example is SDS. Some chemicals have more than one property, such as Triton X-100, which is a detergent but also is an alcohol. Detergents are more diversified and can be alcohol (Brij 35), ester (Tween 20) or organic salt (SDS).

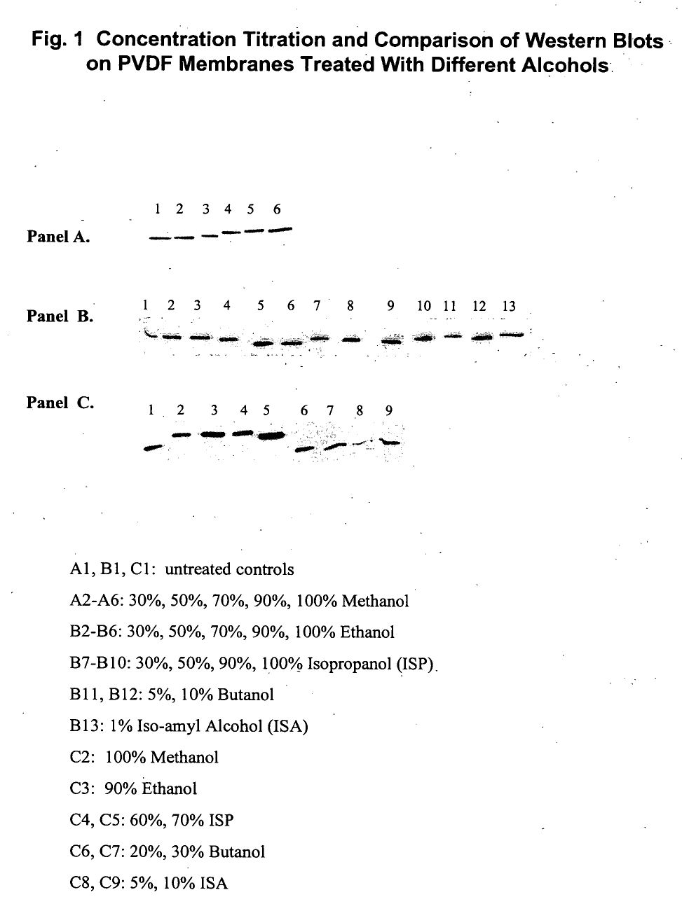

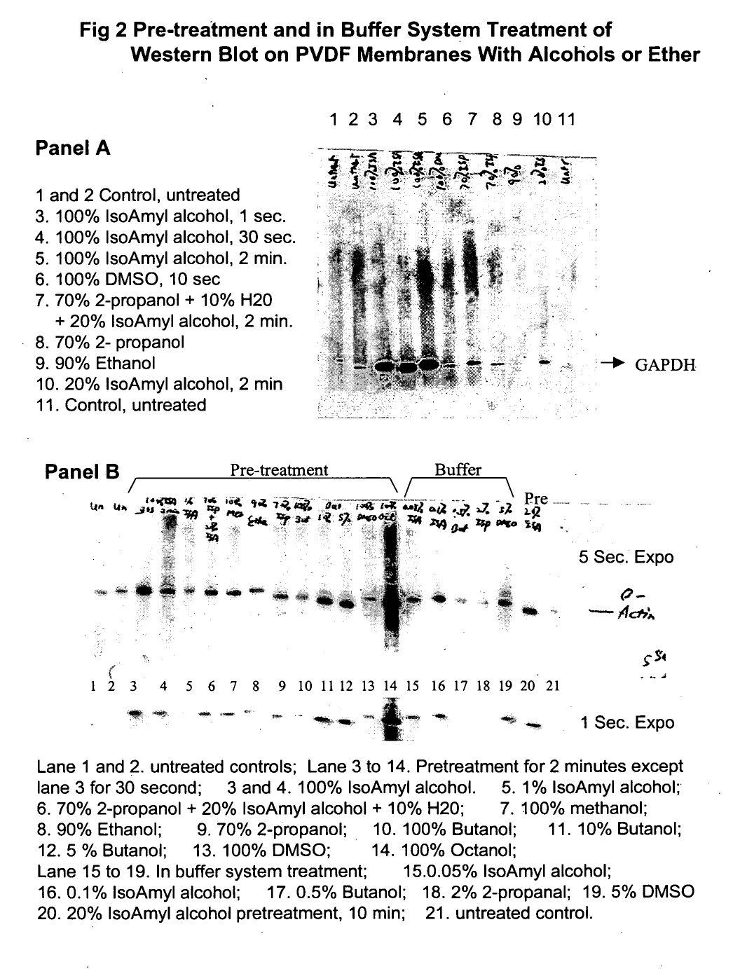

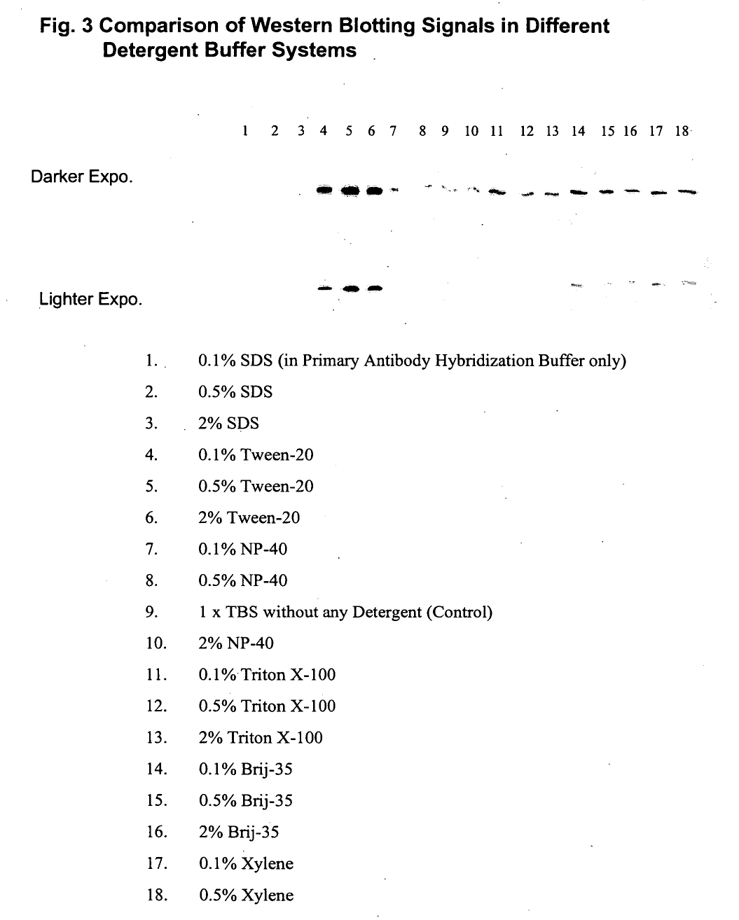

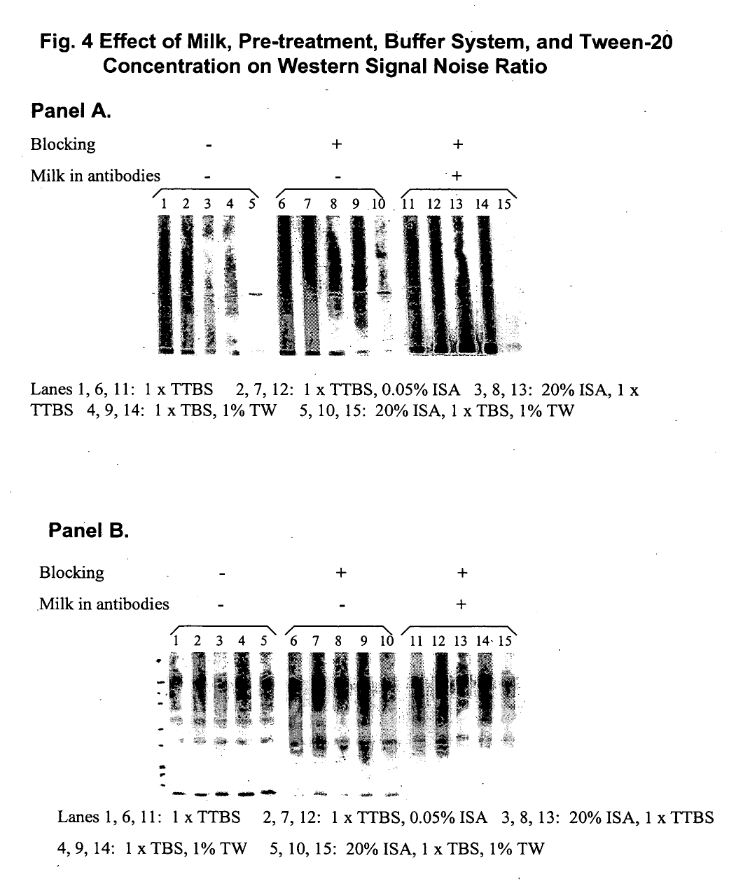

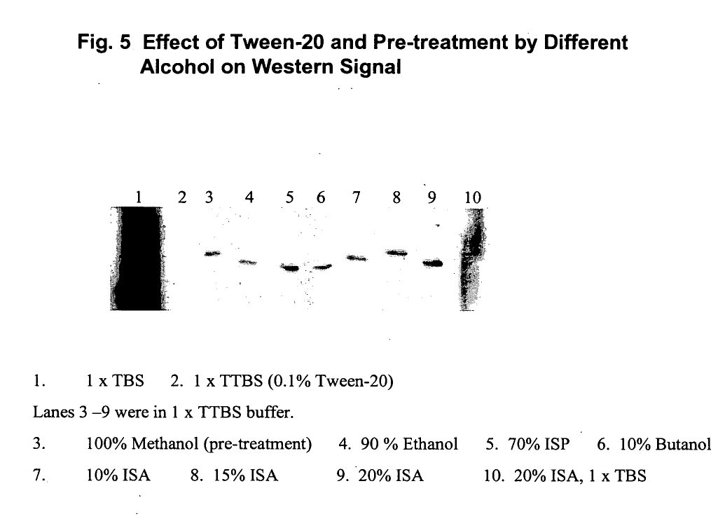

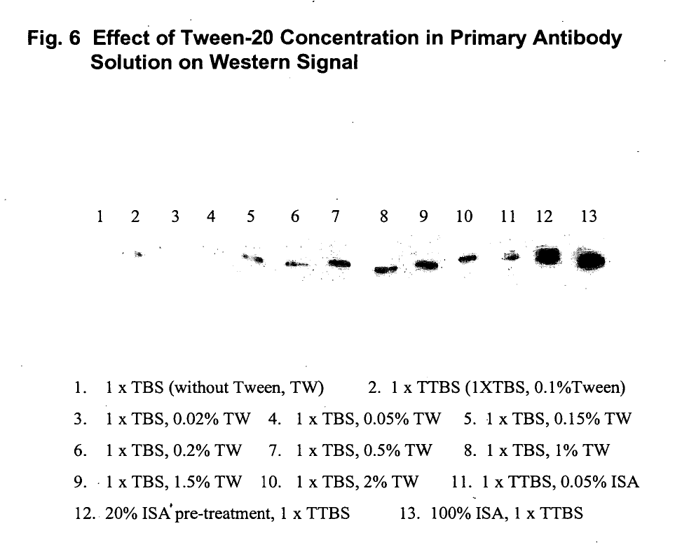

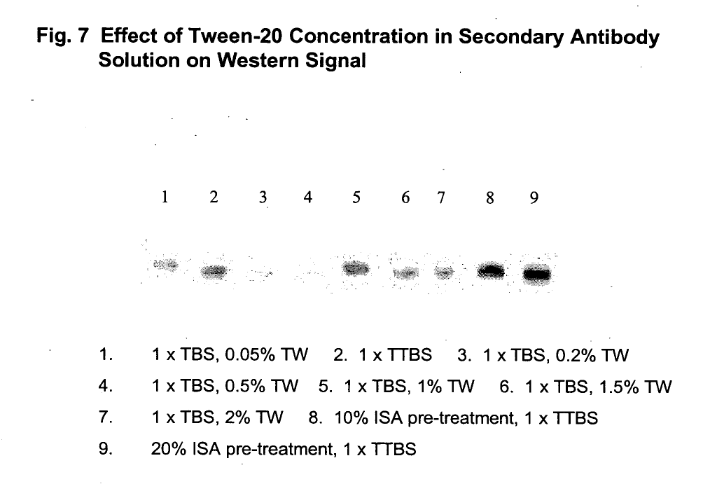

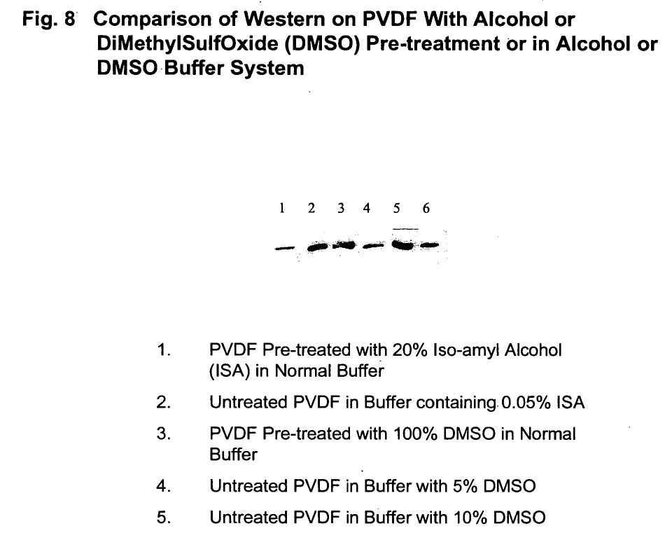

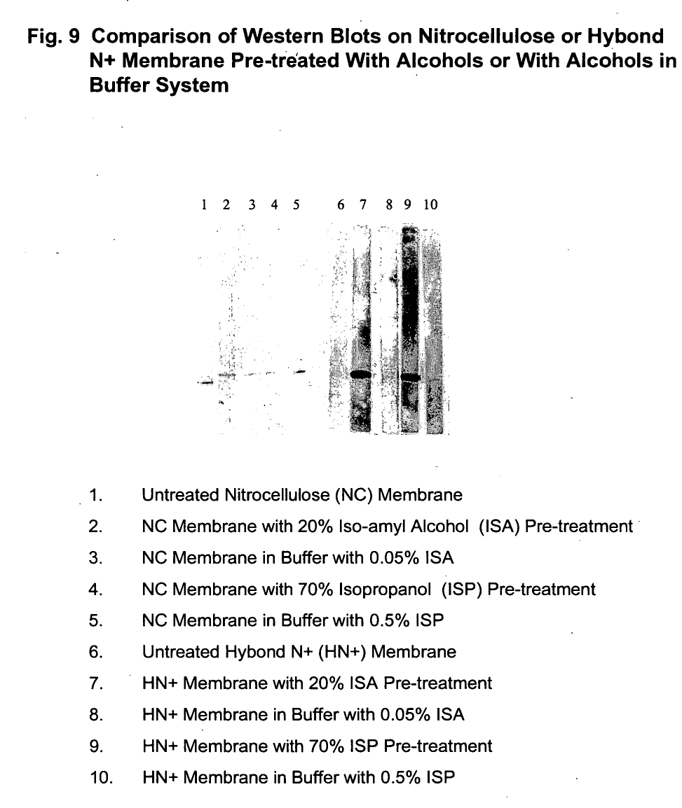

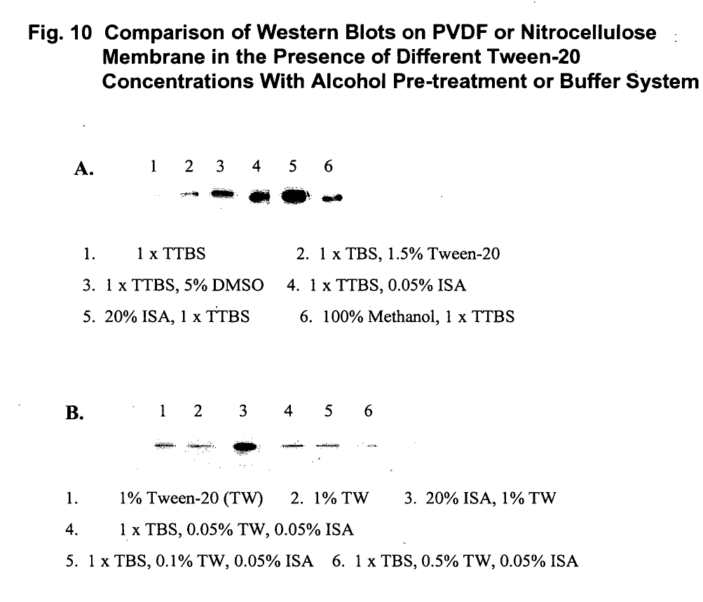

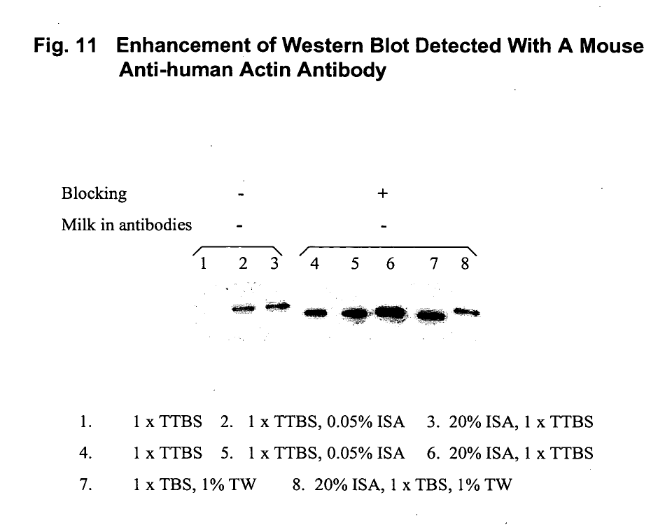

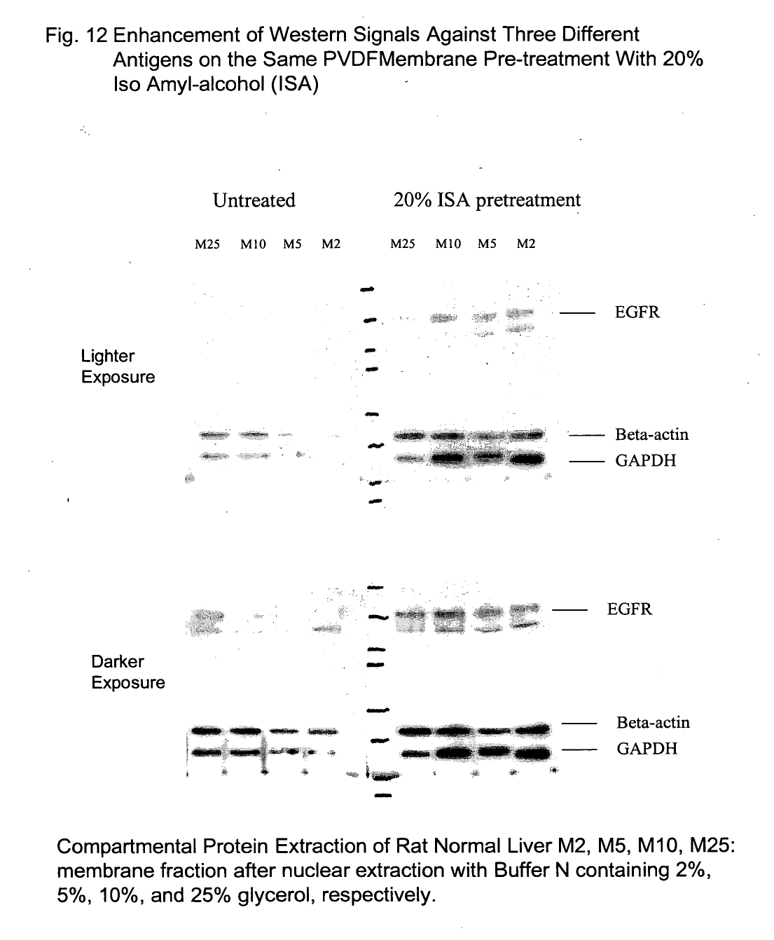

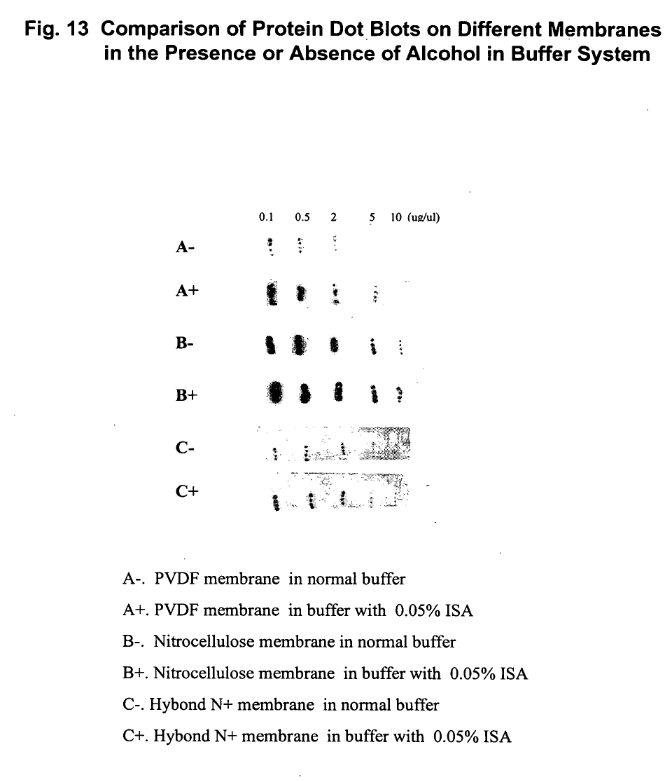

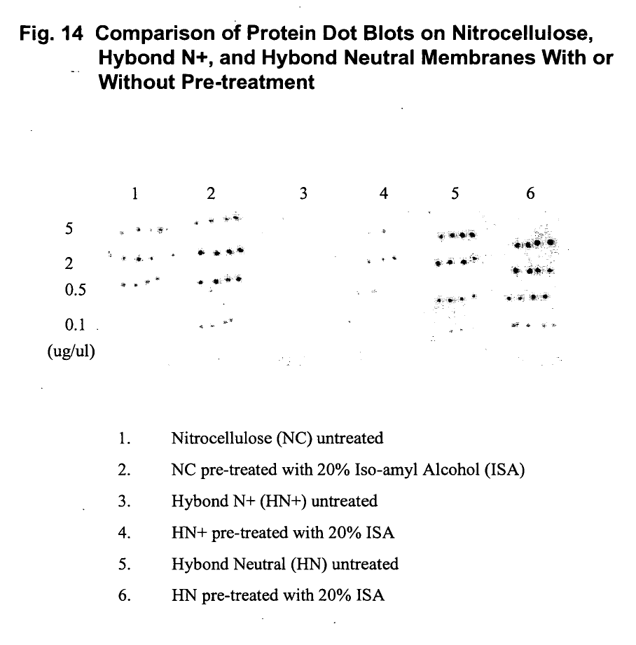

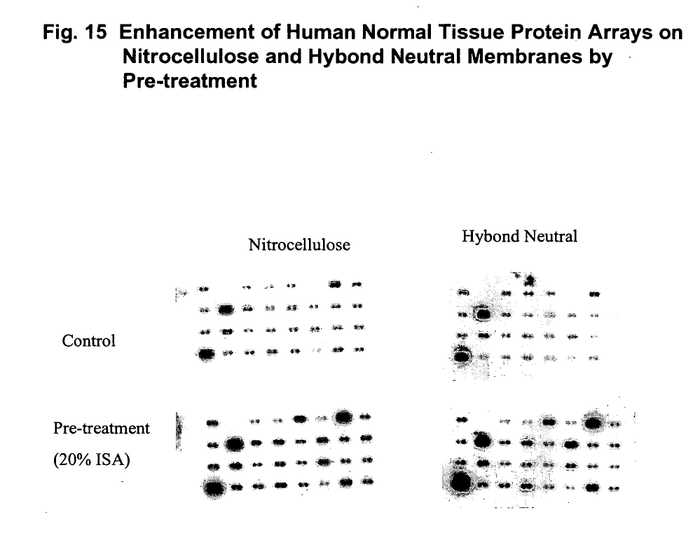

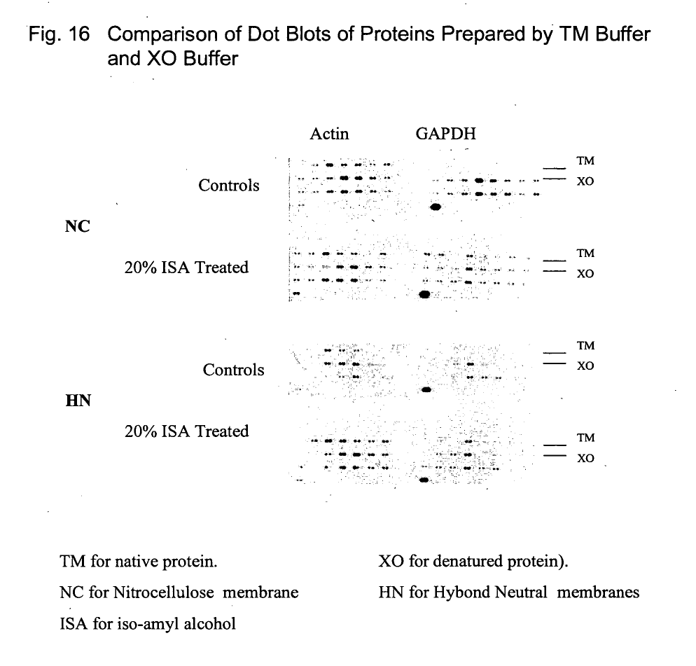

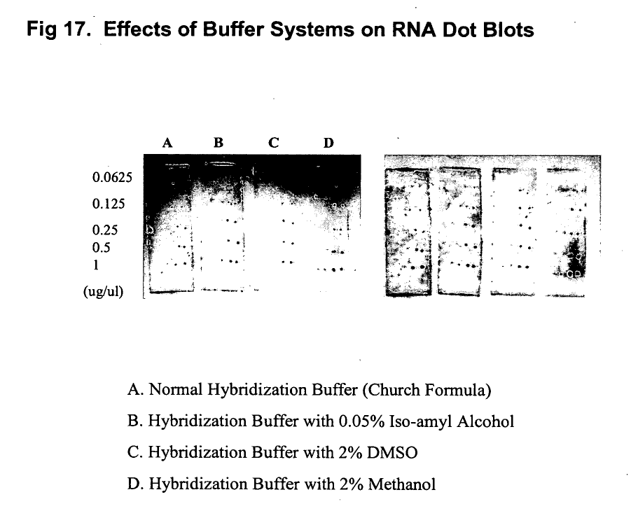

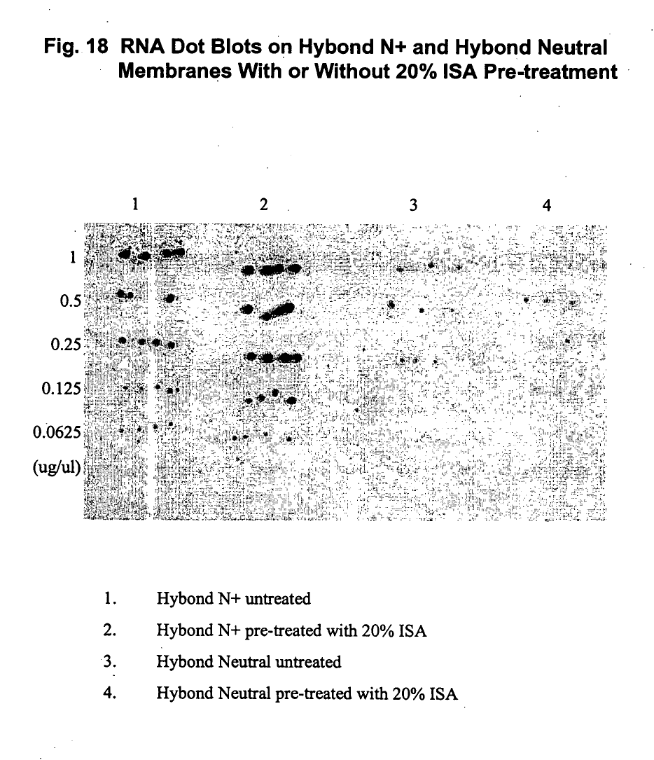

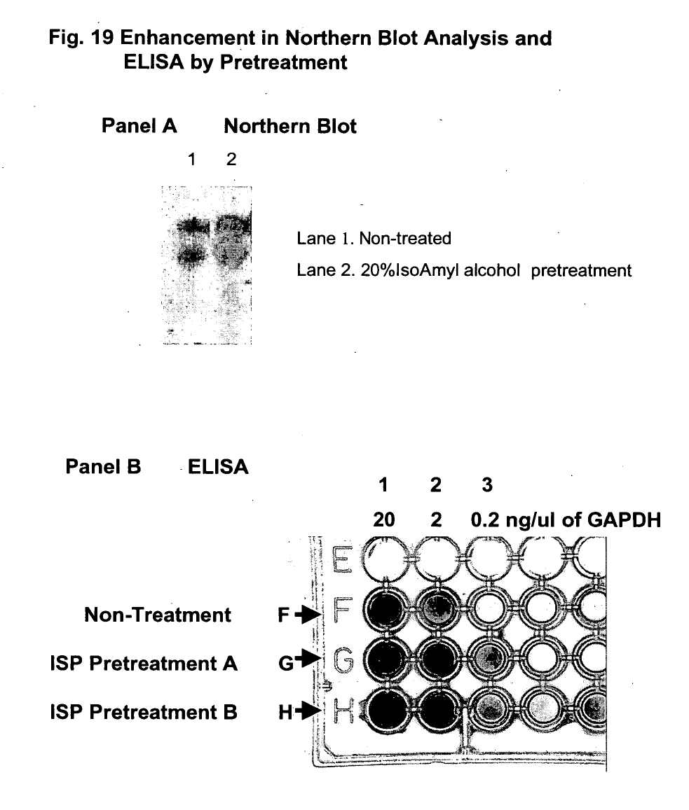

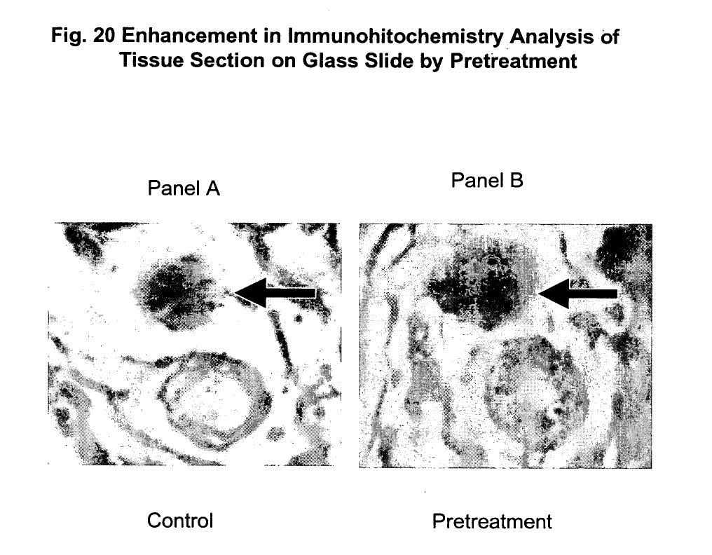

FIGS. 1 and 2 show enhanced interactions (binding) between protein antigens and antibodies, in which protein antigens were treated before or during interactions between protein antigens and antibody. Enhancing efficiencies are different among selected alcohol samples. Pre-treatment with 1-octanol enhances interactions (binding) between protein antigens and antibodies with the strongest signal, and IsoAmyl alcohol and 1-butanol also show substantial enhancing activities as shown in FIG. 2. Brij 35 and Triton X-100 which are considered as alcohols with longer hydrocarbon chain did not show any enhancing activities, but inhibit interactions between protein antigens and antibodies as shown in FIG. 3. Most detergents did not enhance interactions (binding) between protein antigens and antibodies except Tween 20 as shown in FIGS. 3, 5 and 6. Tween 20 is considered as an ester, although it is usually used as a very mild detergent. Dimethylsulfoxide (DMSO) as an ether can enhance interactions (binding) between protein antigens and antibodies when applied in the buffer system as shown in FIGS. 2 and 8. Xylenes (Hydrocarbons in FIG. 3), bovine serum albumin and non-fat dry milk (Proteins in FIG. 4), and SDS (Organic salt in FIG. 3) did not show enhancing activities, or even inhibited the interactions between protein antigens and antibodies as shown in FIGS. 2 and 4. The abilities for organic chemicals or bipolar reagent to enhance interactions between molecules are summarized in Table 1 wherein examples of different organic chemicals or bipolar reagents in pre-treatment for Western blotting are listed and wherein some of alcohols, ester, ether and detergents at appropriate concentrations are shown to enhance interactions among or between molecules. Both pretreatment and treatment in buffer system can enhance interactions among or between molecules. Highest enhancing ability from 1-octanol is about 100 folds higher than control without enhancing treatment.

As shown in Table 1, alcohol family has the most chemicals that can enhance the interactions between protein antigens and antibodies. Ester and ether can also enhance the interactions between protein antigens and antibodies. It is indicated that bipolar organic reagents with less charges and less detergent activity could be the candidates to enhance the interactions between protein antigens and antibodies because bipolar organic reagents can be compatible with hydrophilic or hydrophobic components on protein molecules. Appropriate concentrations of organic chemicals to enhance the interaction of molecules are varied substantially depending on organic chemicals chosen and timing of treatment, such as pretreatment or in buffer system treatment. It can be 0.05% of isoAmyl alcohol in buffer system treatment, or 100% of 1-octanol in pretreatment. It is hard to determine exactly what kinds of organic chemicals have the best enhancing activities. I-octanol can enhance 100 times of the interaction between molecules with higher background, while isoAmyl alcohol can enhance 20 to 50 times of the interaction between molecules with lower background.

TABLE 1
Preferred concentrations and enhancing fold of selected organic
chemicals or bipolar reagents in pre-treatment or
in buffer system treatment for Western Blotting

| | | | | Typical |
|---|---|---|---|---|
| Reagent | Reagent | Preferred | | Enhancing |
| Type | Name | Concentration | | Fold |
| Alcohol | Methanol | 100% | (Pretreatment) | 1 |
| Alcohol | Ethanol | 90% | (Pretreatment) | 1 |
| Alcohol | 2-propanol | 70% | (Pretreatment) | 1-6 |
| Alcohol | 1-Butanol | 10% | (Pretreatment) | 20 |
| Alcohol | IsoAmyl | 20% | (Pretreatment) | 20-80 |
| | Alcohol | to 100% | | |
| Alcohol | IsoAmyl | 0.05-0.1% | (in buffer) | 10-15 |
| | Alcohol | | | |
| Alcohol | 1-Octanol | 100% | (Pretreatment) | 100 |
| Alcohol/ | Triton | 0.5% | (in buffer) | No enhance |
| Detergent | X-100 | | | |
| Ester/ | Tween 20 | 1% | (in buffer) | 5-10 |
| Detergent | | | | |
| Ether | DMSO | 100% | (Pretreatment) | 1 |
| Ether | DMSO | 5% | (in buffer) | 10 |
| Hydrocarbons | Xylenes | 100% | (Pretreatment) | No enhance |
| Protein | Dry milk | 5% | (in buffer) | No enhance |
| Organic Salt/ | SDS | 0.5% | (in buffer) | No enhance |
| Detergent | | | | |

There are a few hypotheses for organic chemicals to enhance interactions between molecules: 1) organic chemicals may reconfigure the three-dimensional structure of epitopes to fit counterpart molecules; 2) organic chemicals may unmask some noncovalent bonds such as hydrogen bonds, vander Waals force, Coulombic interaction and hydrophobic bonds to increase the affinity of the interactions between molecules; 3) organic chemicals acting as surfactant may restore the properties of molecules surface, such as positive or negative charge, hydrophilic or hydrophobic status to match the counterpart molecules; and 4) organic chemicals as bipolar reagents may help antibodies to penetrate into hydrophobic supporting materials permitting their interactions with antigens.

This invention supports the first three hypotheses, but not favors the last hypothesis. Organic chemicals can improve the lower level of interaction between “native” protein (TM) and GAPDH antibodies into the same level as the interaction between “denatured” protein (XO) and GAPDH antibodies in FIG. 16. This data indicates that organic chemicals reconfigure the structure of “native” proteins into the structure of “denatured” proteins, in which structure of “denatured” proteins has correct epitopes to bind with GAPDH antibody. Another data support structural reconfiguration of molecules by organic chemicals is that organic chemicals have more enhancing effect on the interactions between proteins and antibodies than the interactions between nuclear acid and nuclear acid as comparisons of FIG. 2 with FIGS. 17, 18 and 19. In hybridization nuclear acids are denatured into linear structure and may not have very complicated three-dimensional structures or epitopes like protein did. Thus, there are not so many spaces for organic chemicals to enhance the interaction between nuclear acids.

The hypothesis that organic chemicals may unmask some noncovalent bonds such as hydrogen bonds, vander Waals force, Coulombic interaction and hydrophobic bonds to increase the affinity of the interactions between molecules is supported by the data in this invention. The data of stronger enhancing abilities of interaction between protein and antibodies than the interaction between nuclear acids as comparing FIG. 2 with FIGS. 17, 18 and 19 indicates that the extra binding force may be unmasked. The interaction between nuclear acids is mainly through hydrogen bonds whereas the interactions between protein and antibodies involves many other bonds, such as vander Waals force, Coulombic interaction and, especially, hydrophobic bonds. It is highly possible that organic chemicals unmask some noncovalent bonds, especially hydrophobic bonds, to enhance the interactions between molecules.

The hypothesis that organic chemicals acting as surfactant may restore the properties of molecules surface, such as positive or negative charge, hydrophilic or hydrophobic status to match the counterpart molecules is supported by the date in FIG. 3. Tween 20 is a very mild surfactant or detergent and is an ester too. Its bipolar activity and surfactant activities are not as strong as alcohols with longer hydrocarbons backbone such as Triton X-100, and are much weaker than organic salt such as SDS. It can restore the properties of molecules surface, such as positive or negative charge, hydrophilic or hydrophobic status to match the counterpart molecules. The other supporting data indicates that Tween 20 has restored different epitopes or in different ways to restore the same epitopes as alcohols have reconfigured such that the enhancing effects of alcohols can be added onto the enhancing effect of Tween 20 as shown in FIGS. 5 and 6.

The last hypothesis that organic chemicals as bipolar reagents may help antibodies to penetrate into hydrophobic supporting materials permitting their interaction with antigens is not supported by data in this invention. Although this hypothesis may explain the enhancing effect on hydrophobic membrane (PVDF), the similar enhancing effects are also presented on other hydrophilic membrane (NC or Nylon) as shown in FIGS. 9, 10 15 and 16. Pretreatment by DMSO has less enhancing effect that in buffer system treatment with DMSO as shown in FIGS. 2 and 8, which also indicates that the penetration hypothesis may not be correct.

The mechanisms for organic chemicals to enhance interactions between molecules can be any one of first three hypotheses. Organic chemicals may reconfigure the three dimensional structure of epitopes; organic chemicals may unmask extra noncovalent bonds; and organic chemicals may restore the charge, hydrophilic or hydrophobic status of molecules. Furthermore, the mechanisms can be any combinations of first three hypotheses or all of first three hypotheses. This invention has at least shown one example in combinations of different mechanisms, such that enhancing effects of alcohols can be added onto the enhancing effect of Tween 20 as shown in FIGS. 5 and 6. In this example of combinational mechanisms, Tween 20 has restored different epitopes from the epitopes reconfigured by alcohols, or Tween 20 has different mechanism to restore the same epitopes as alcohols have reconfigured.

### EXAMPLE 1

As shown in Table 1 alcohol family has the most chemicals that can enhance the interactions between protein antigens and antibodies. Ester and ether can also enhance the interactions between protein antigens and antibodies. It is indicated that bipolar organic reagents with less charges and less detergent activity could be the candidates to enhance the interactions between protein antigens and antibodies because bipolar organic reagents can be compatible with hydrophilic or hydrophobic components on protein molecules. Appropriate concentrations of organic chemicals to enhance the interaction of molecules are varied substantially depending on organic chemicals chosen and timing of treatment, such as pretreatment or in buffer system treatment. It can be 0.05% of isoAmyl alcohol in buffer system treatment, or 100% of 1-octanol in pretreatment. It is hard to determine exactly what kinds of organic chemicals have the best enhancing activities. I-octpanol can enhance 100 times of the interaction between molecules with higher background, while isoAmyl alcohol can enhance 20 to 50 times of the interaction between molecules with lower background.

### EXAMPLE 2

Supporting materials for molecules may not be relevant to the enhancing activities of the interactions between molecules, such as antibodies and protein antigens, or nuclear acid and nuclear acid with treatment by organic chemicals. No matter what kinds of supporting materials the molecules were immobilized onto, the enhancing activities will be present when molecules were treated with appropriate organic chemicals. The supporting material can be membrane (FIGS. 12, 13, and 14), glass slide (FIG. 20) or natural biological materials (FIG. 20), or plastics (FIG. 19). The membrane can be made from nylon (FIGS. 9, 13, 14, 15, and 16), nitrocellulose (FIGS. 9, 10, 13, 14, 15, and 16) or polyvinylidene difluoride (PVDF) (FIG. 1-8, and 12). Therefore, supporting materials for molecules may not be a direct target for treatment by organic chemicals to enhance the interactions between antibodies and protein antigens. Table 2 shows the enhancing function of organic chemicals or bipolar reagents on Interactions among or between different molecules on different supporting materials wherein the enhancing function of pre-treatment and in buffer system treatment by organic chemicals or bipolar reagents on Western blot, protein dot blot, tissue sections, ELISA, Northern blot and RNA dot blot, on different supporting materials is summarized according to the invention. Pre-treatment could enhance signals in all six assays; treatment in buffer system can enhance three of all six assays. Supporting materials may not affect enhancing function of organic chemicals or bipolar reagents.

### EXAMPLE 3

Molecules immobilized onto supporting materials are direct targets for treatment by organic chemicals to enhance the interactions between molecules, such as antibodies and protein antigens, or nuclear acid and nuclear acid with treatment with organic chemicals. The example molecules used in this invention are proteins and nuclear acid. The enhancing effect can be achieved on different molecules under different status. The proteins can be native protein extracted by a less stringency method (TM method) or denature protein extracted by a harsh method (XO method) as shown in FIGS. 12 and 16. Proteins can be denatured by SDS-PAGE and transferred on to a supporting materials (FIGS. 9, 10 and 12), or can be relatively native and spotted on to supporting materials (FIGS. 14, 15 and 16). Proteins can even stay inside tissue without extraction (FIG. 20). RNA can also be denatured by denaturing RNA gel electrophoresis and transferred on to a supporting materials (FIG. 19), or can be relatively native and spotted on to supporting materials (FIGS. 17 and 18).

TABLE 2
Enhancing function of organic chemicals or bipolar reagents on
interactions among or between different molecules on
different supporting materials

| | | Protein | | | | Nuclear | |
|---|---|---|---|---|---|---|---|
| Organic | Supporting | | | Tissue | | Acids | |
| Chemicals | materials | Blot | Dot | section | ELISA | Blot | Dot |
| Pre- | Nitro- | Yes | Yes | | | | |
| Treatment | cellulose | | | | | | |
| Alcohols | Nylon | Yes | Yes | | | Yes | Yes |
| Ester, | PVDF | Yes | | | | | |
| Ether | SiO2 | | | Yes | | | |
| Detergent | (Glass) | | | | | | |
| | Tissue | | | Yes | | | |
| | matrixes | | | | | | |
| | PVC (wells) | | | | Yes | | |
| In Buffer | Nitro- | Yes | Yes | | | | |
| System | cellulose | | | | | | |
| Treatment | Nylon | Yes | Yes | | | | Yes |
| Alcohols | PVDF | Yes | | | | | |
| Ester, | SiO2 | | | | | | |
| Ether | (Glass) | | | | | | |
| Detergent | Tissue | | | | | | |
| | matrixes | | | | | | |
| | PVC (wells) | | | | | | |

### EXAMPLE 4

The counterpart molecules bound to molecules in Example 3 may not relate directly to the enhancing activities of the interactions between molecules, such as antibodies and protein antigens, or nuclear acid and nuclear acid with treatment by organic chemicals. The example counterpart molecules used in this invention for interaction between molecules are antibodies or nuclear acid, which are the probes frequently used in life science discovery and diagnosis of disorders. Antibodies anti different antigens, such as antibodies anti cellular protein (GAPDH) from Chemicon, anti structure protein (Actin) from Sigma and anti membrane protein (EGFR) from Santa Crus Biotechnology are tested as shown in FIGS. 2 and 12. These antibodies are derived from different hosts. Antibodies anti GAPDH and Actin are from mouse and antibodies anti. EGFR is from rabbit. Antibodies anti GAPDH and Actin are monoclonoal antibodies and antibodies anti EGFR is polyclonal antibody. Antigens or immunogens used to raise antibody are different. GAPDH is from rabbit muscle; Actin is N-terminal peptide and EGFR is C-terminal peptide. These antibodies may have preference to binding on native proteins or denatured proteins. Probe used in RNA analysis is made from human actin cDNA products amplified and labeled by polymerase chain reaction. This probe is a housekeeping gene labeled by very common method. Therefore, the counterpart molecules bound to molecules in Example 3 are routine materials that may not play significant role in enhancing activities of the interactions between molecules, such as antibodies and protein antigens, or nuclear acid and nuclear acid with treatment by organic chemicals.

### EXAMPLE 5

Molecules immobilized onto supporting materials can be treated in different ways to enhance the interactions between molecules, such as antibodies and protein antigens, or nuclear acid and nuclear acid with treatment by organic chemicals. The different ways include timing, stage, solo or mixture of organic chemicals, and combinations of different timing and different organic chemicals. Molecules immobilized onto supporting materials can be treated in different timing, such as pretreated in organic chemicals for 1 second to 10 minutes before interactions of molecules, or can be in buffer system treatment for overnight or three days during interactions of molecules as shown in FIGS. 2, 3, 6, 8, 18, 19 and 20. Treatment at different stages of interaction between molecules, such as at first antibodies incubation or at both first antibodies and secondary antibodies incubation, do not show significant different in enhancing activities when molecules are treated in buffer system with Tween 20 as shown in FIGS. 6 and 7. Molecules immobilized onto supporting materials can be treated with solo or mixture of organic chemicals to enhance the interactions between molecules as shown in FIGS. 2, 6, 10, 11, and 13. The combinations of different timing and different organic chemicals can synergy the enhancing activities of interactions between molecules, such as pretreatment of molecules with IsoAmyl alcohol and in buffer system treatment with Tween 20 as shown in FIGS. 5 and 6.

### EXAMPLE 6

There are three hypotheses for organic chemicals to enhance interactions between molecules: 1) organic chemicals may reconfigure the three-dimensinal structure of epitopes to fit counterpart molecules; 2) organic chemicals may unmask some noncovalent bonds such as hydrogen bonds, vander Waals force, Coulombic interaction and hydrophobic bonds to increase the affinity of the interactions between molecules; 3) organic chemicals acting as surfactant may restore the properties of molecules surface, such as positive or negative charge, hydrophilic or hydrophobic status to match the counterpart molecules;

The mechanisms for organic chemicals to enhance interactions between molecules can be any one of the three hypotheses. Organic chemicals may reconfigure the three dimensional structure of epitopes; organic chemicals may unmask extra noncovalent bonds; and organic chemicals may restore the charge, hydrophilic or hydrophobic status of molecules. Furthermore, the mechanisms can be any combinations of three hypotheses or all of three hypotheses. This invention has at least shown one example illustrating that the enhancing effects can be caused by combinations of different mechanisms, such that, for example, the enhancing effects of alcohols can be added onto the enhancing effect of Tween 20 as shown in FIGS. 5 and 6. In this example of combinational mechanisms, Tween 20 has restored different epitopes from the epitopes reconfigured by alcohols, or Tween 20 has different mechanism to restore the same epitopes as alcohols have reconfigured.

In summary, this invention discovers a novel method to increase or enhance abilities of interaction between molecules, especially biomolecules, such as proteins or nuclear acid, by optimizing environment condition for interactions. Interactions between molecules are determined by the intrinsic bonds (the properties of components on molecules, distance between components, or conformational structures of epitopes), valences between molecules, three-dimensional structure of multiple binding sites on molecules to form multimeric complexes. The properties of components on molecules, potential valences between molecules and multiple binding sites on molecules are given by nature in the molecules, but optimization in environment condition for interactions such as hydrophilic or hydrophobic conditions by organic chemicals or bipolar reagents may optimize distance between components on molecules, conformational structures of epitopes and three-dimensional structure of multiple binding sites on molecules, therefore increase capabilities of interactions between molecules.

The invention has been described using exemplary preferred embodiments. However, for those skilled in this field, the preferred embodiments can be easily adapted and modified to suit additional applications without departing from the spirit and scope of this invention. Thus, it is to be understood that the scope of the invention is not limited to the disclosed embodiments. On the contrary, it is intended to cover various modifications and similar arrangements based upon the same operating principle. The scope of the claims, therefore, should be accorded the broadest interpretations so as to encompass all such modifications and similar arrangements.

We claim:

1. A method for enhancing interactions among or between molecules in a reaction environment comprising the steps of: a) providing the molecules with organic chemicals or bipolar reagents in the reaction environment; and b) treating the molecules with said organic chemicals or bipolar reagents for a period of time sufficient to induce or enhance interactions among said molecules.

2. The method of claim 1, wherein the molecules are cellular molecules capable of interacting or binding to each other, such as proteins and antibodies, or nuclear acid and nuclear acid.

3. The method of claim 1, wherein the step of inducing or enhancing interactions including binding of the molecules to each other.

4. The method of claim 1, wherein the organic chemicals or bipolar reagents are chemicals containing at least carbon and hydrogen and showing function of enhancing interactions among or between molecules, such as alcohols, detergents, esters, and ethers.

5. The method of claim 1, wherein the treatments by organic chemicals or bipolar reagents can take place before the interactions among or between the molecules, or during interactions among or between molecules.

6. The method of claim 1, wherein the treatments by organic chemicals or bipolar reagents further comprises the step of treating the molecules to be interacted with other counterpart molecules.

7. The method of claim 1, wherein the molecules can be in a denatured or in a native status.

8. The method of claim 1 may comprise the step of immobilizing the molecules on supporting materials or not.

9. The method of claim 8, wherein the molecule immobilizing step includes electrical transfer, capillary transfer, direct spotting, or fixation.

10. The method of claim 8, wherein the supporting materials can be made into different shapes, formats or status, such as membrane, multiple well plates, sheet, fiber, column, beads, and glass plate.

11. The method of claim 8, wherein the supporting materials can be made from organic or non-organic chemicals, such as nylon, nitrocellulose, polyvinylidene difluoride (PVDF), polyvinyl chloride (PVC), plastic, silicon, and natural biological materials.

12. The method of claim 1, wherein the step of treating the molecules by organic chemicals or bipolar reagents includes treatment of the supporting materials to enhance interactions among or between molecules.

13. The method of claim 4, wherein the alcohols showing function of enhancing interactions among or between molecules are methanol, ethanol, 2-propanol, 1-butanol, IsoAmyl alchohol, and 1-octanol.

14. The method of claim 4, wherein the detergent showing function of enhancing interactions among or between molecules at least is Tween 20.

15. The method of claim 4, wherein the ester showing function of enhancing interactions among or between molecules at least is Tween 20.

16. The method of claim 4, wherein the ether showing function of enhancing interactions among or between molecules at least is Dimethylsulfoxide (DMSO)

17. The method of claim 5, wherein the step of treating the molecules with organic chemicals or bipolar reagents before the interactions among or between molecules includes immersing the molecules into organic chemicals or bipolar reagents, or into a media containing organic chemicals or bipolar reagents for 1 second to 10 minutes.

18. The method of claim 5, wherein the step of treating the molecules with organic chemicals or bipolar reagents during interactions among or between molecules includes immersing molecules into organic chemicals or bipolar reagents, or into a media containing organic chemicals or bipolar reagents for the period of interactions among or between molecules.

19. The method of claim 4, wherein the organic chemicals or bipolar reagents used in treatments can be a single organic chemical or a bipolar reagent, or can be mixture of organic chemicals or bipolar reagents.

20. The method of claim 5, wherein treatments by organic chemicals or bipolar reagents can be a combination of before and during the interactions among or between molecules.

21. The method of claim 1, wherein the organic chemicals or bipolar reagents can be 100% pure, or can be a mixture in any percentage with other chemicals or water.

22. The method of claim 6, wherein the molecules treated by organic chemicals can interact with other counterpart molecules such as antibodies and nuclear acids.

23. The method of claim 22, wherein the antibodies can be monoclonal or polyclonal antibodies.

24. The method of claim 22, wherein the antibodies can be derived at least from the hosts of mouse or rabbit.

25. The method of claim 22, wherein the immunogens or antigens used to raise the antibodies can be at least peptides or proteins.

26. The method of claim 22, wherein the antibodies can bind preferably to either native antigens or denatured antigens.

27. The method of claim 22, wherein the antibodies can be raised from nuclear acids such as DNA or RNA.

28. The method of claim 1, wherein the step of enhancing interactions among or between molecules with treatments of organic chemicals or bipolar reagents includes reconfiguring structures of epitopes on molecules, unmasking noncovalent bonds between molecules, or restoring charges, hydrophilic or hydrophobic status of molecules.

29. A product for enhancing interactions among or between molecules in a reaction environment comprising a) suitable organic chemicals or bipolar reagents prepared in the reaction environment; and b) molecules of interest added to said organic chemicals or bipolar reagents for treatment for a period of time sufficient to induce or enhance interactions among said molecules of interest.

30. The product of claim 29, wherein the molecules of interest are cellular molecules capable of interacting or binding to each other, such as proteins and antibodies, or nuclear acid and nuclear acid.

31. The product of claim 29, wherein the organic chemicals or bipolar reagents are chemicals containing at least carbon and hydrogen and showing function of enhancing interactions among or between molecules, such as alcohols, detergents, esters, and ethers.

32. The product of claim 29, wherein the treatments by organic chemicals or bipolar reagents can take place before the interactions among or between the molecules, or during interactions among or between molecules.

33. The product of claim 29, wherein the treatments by organic chemicals or bipolar reagents further comprises treating the molecules of interest to be interacted with other counterpart molecules.

34. The product of claim 29, wherein the molecules of interest can be in a denatured or in a native status.

35. The product of claim 29 further comprises supporting materials to provide immobilization of the molecules of interest.

36. The product of claim 35, wherein the supporting materials that provides the immobilization of the molecules of interest are applicable to electrical transfer, capillary transfer, direct spotting, or fixation.

37. The product of claim 35, wherein the supporting materials can be made into different shapes, formats or status, such as membrane, multiple well plates, sheet, fiber, column, beads, and glass plate.

38. The product of claim 35, wherein the supporting materials can be made from organic or non-organic chemicals, such as nylon, nitrocellulose, polyvinylidene difluoride (PVDF), polyvinyl chloride (PVC), plastic, silicon, and natural biological materials.

39. The product of claim 35, wherein the supporting materials can be treated by organic chemicals or bipolar reagents to enhance interactions among or between molecules of interest.

40. The product of claim 31, wherein the alcohols showing function of enhancing interactions among or between molecules are methanol, ethanol, 2-propanol, 1-butanol, IsoAmyl alchohol, and 1-octanol.

41. The product of claim 31, wherein the detergent showing function of enhancing interactions among or between molecules at least is Tween 20.

42. The product of claim 31, wherein the ester showing function of enhancing interactions among or between molecules at least is Tween 20.

43. The product of claim 31, wherein the ether showing function of enhancing interactions among or between molecules at least is Dimethylsulfoxide (DMSO)

44. The product of claim 32, wherein the treatment of the molecules of interest with organic chemicals or bipolar reagents before the interactions among or between molecules includes immersing the molecules of interest into organic chemicals or bipolar reagents, or into a media containing organic chemicals or bipolar reagents for 1 second to 10 minutes.

45. The product of claim 32, wherein the treatment of the molecules with organic chemicals or bipolar reagents during interactions among or between molecules includes immersing molecules into organic chemicals or bipolar reagents, or into a media containing organic chemicals or bipolar reagents for the period of interactions among or between molecules.

46. The product of claim 31, wherein the organic chemicals or bipolar reagents used in treatments can be a single organic chemical or a bipolar reagent, or can be mixture of organic chemicals or bipolar reagents.

47. The product of claim 31, wherein treatments by organic chemicals or bipolar reagents can be a combination of before and during the interactions among or between the molecules of interest.

48. The product of claim 29, wherein the organic chemicals or bipolar reagents can be 100% pure, or can be a mixture in any percentage with other chemicals or water.

49. The product of claim 33, wherein the molecules of interest treated by organic chemicals can interact with other counterpart molecules such as antibodies and nuclear acids.

50. The product of claim 49, wherein the antibodies can be monoclonal or polyclonal antibodies.

51. The product of claim 50, wherein the antibodies can be derived at least from the hosts of mouse or rabbit.

52. The product of claim 49, wherein the immunogens or antigens used to raise the antibodies can be at least peptides or proteins.

53. The product of claim 49, wherein the antibodies can bind preferably to either native antigens or denatured antigens.

54. The product of claim 49, wherein the antibodies can be raised from nuclear acids such as DNA or RNA.

55. The product of claim 29, wherein the enhancement of interactions among or between molecules with treatments of organic chemicals or bipolar reagents includes reconfiguring structures of epitopes on molecules, unmasking noncovalent bonds between molecules, or restoring charges, hydrophilic or hydrophobic status of molecules.
